(19) United States
(12) Patent Application Publication
(10) Pub. No.: US 2024/0424312 A1
(43) Pub. Date: Dec. 26, 2024

(54) IMPLANTABLE DEVICE TO EXTERNAL DEVICE COMMUNICATION USING DIRECT-SEQUENCE SPREAD SPECTRUM

(71) Applicant: Cardiac Pacemakers, Inc., St. Paul, MN (US)

(72) Inventor: Michael John Shea, Eagan, MN (US)

(21) Appl. No.: 18/753,125

(22) Filed: Jun. 25, 2024

Related U.S. Application Data
(60) Provisional application No. 63/523,164, filed on Jun. 26, 2023.

(51) Int. Cl. A61N 1/372; A61N 1/02; A61N 1/05; A61N 1/375

(52) U.S. Cl. CPC A61N 1/37254; A61N 1/025; A61N 1/056; A61N 1/37264; A61N 1/3727; A61N 1/37512; A61N 1/3754; A61N 1/3758

(57) ABSTRACT

Embodiments herein relate to implantable medical devices. In an embodiment, an implantable medical device is included having a biocompatible housing, a control circuit that can disposed within the biocompatible housing, and a wireless communications transceiver. The wireless communications transceiver can be configured to communicate wirelessly through a first channel using a standard BLUETOOTH protocol (non-DSSS) and communicate wirelessly through a second channel using a direct sequence spread spectrum (DSSS) protocol over a BLUETOOTH frequency band. Other embodiments are also included herein.

This application claims the benefit of U.S. Provisional Application No. 63/523,164, filed Jun. 26, 2023, the content of which is herein incorporated by reference in its entirety.

### FIELD

Embodiments herein relate to medical device systems including features to enable wireless communications between components thereof.

### BACKGROUND

Many implantable medical devices have functions that are lifesaving, important to patient health, important to diagnosis, or multiple of these functions. Many implantable medical devices can wirelessly communicate with external devices outside of the body. Wireless communication with implantable medical devices can facilitate programming of the implantable medical device, status checks of the implantable medical device, transmission of patient data, and initiating enhanced data collection at important moments in time, such as when a patient is experiencing symptoms. Methods and systems for bidirectional wireless communication between an implantable medical device and external devices are needed that are both reliable and secure.

### SUMMARY

In a first aspect, an implantable medical device can be included having a biocompatible housing, a control circuit that can disposed within the biocompatible housing, and a wireless communications transceiver. The wireless communications transceiver can be configured to communicate wirelessly through a first channel using a standard BLUETOOTH protocol (non-DSSS) and communicate wirelessly through a second channel using a direct sequence spread spectrum (DSSS) protocol over a BLUETOOTH frequency band.

In a second aspect, in addition to one or more of the preceding or following aspects, or in the alternative to some aspects, the wireless communications transceiver can be configured to communicate simultaneously through both the first channel and the second channel.

In a third aspect, in addition to one or more of the preceding or following aspects, or in the alternative to some aspects, the wireless communications transceiver can be configured to communicate wirelessly to a first external device through the first channel and to communicate wirelessly to a second external device through the second channel.

In a fourth aspect, in addition to one or more of the preceding or following aspects, or in the alternative to some aspects, the first external device can be any of a cellular phone, a smart phone, a laptop computer, a tablet-style computer, a desktop computer, and a wearable device.

In a fifth aspect, in addition to one or more of the preceding or following aspects, or in the alternative to some aspects, the second external device can be any of an implantable medical device programmer, an external implantable medical device communicator, and an external medical device.

In a sixth aspect, in addition to one or more of the preceding or following aspects, or in the alternative to some aspects, the wireless communications transceiver can be configured to troubleshoot the first channel by communicating through the second channel or to troubleshoot the second channel by communicating through the first channel.

In a seventh aspect, in addition to one or more of the preceding or following aspects, or in the alternative to some aspects, the DSSS protocol includes encoding communications through the second channel with a spreading sequence such that the communications can only be decoded by devices possessing the spreading sequence.

In an eighth aspect, in addition to one or more of the preceding or following aspects, or in the alternative to some aspects, the wireless communications transceiver can be configured to receive the communications from an external device, wherein the communications include a command to be implemented by the implantable medical device. The control circuit can be configured to implement the command if the communications are encoded with the spreading sequence and not implement the command if the communications are not encoded with the spreading sequence.

In a ninth aspect, in addition to one or more of the preceding or following aspects, or in the alternative to some aspects, the wireless communications transceiver can be configured to receive the communications from an external device, wherein the communications include a command to be implemented by the implantable medical device. The control circuit can be configured to implement the command if the communications is received over the second channel and not implement the command if the communications is received over the first channel.

In a tenth aspect, in addition to one or more of the preceding or following aspects, or in the alternative to some aspects, the implantable medical device includes high-risk data, and wherein the wireless communications transceiver can be configured to communicate the high-risk data through only the second channel.

In an eleventh aspect, in addition to one or more of the preceding or following aspects, or in the alternative to some aspects, the implantable medical device can be a pulse generator and the wireless communications transceiver can be configured to communicate through the first channel to one or more patient owned devices and to communicate through the second channel to any of an implantable medical device programmer or an implantable medical device communications device.

In a twelfth aspect, in addition to one or more of the preceding or following aspects, or in the alternative to some aspects, the implantable medical device can be a pulse generator and the wireless communications transceiver can be configured to communicate through the first channel to one or more patient owned devices and to communicate through the second channel to an external medical device.

In a thirteenth aspect, a medical device system can be included having an implantable medical device. The implantable medical device can include a control circuit, and a wireless communications transceiver. The wireless communications transceiver can be configured to communicate wirelessly through a first channel using a standard BLUETOOTH protocol and communicate wirelessly through a second channel using a DSSS protocol over a BLUETOOTH frequency band. The medical device system can include a first external device, wherein the first external device can be configured to communicate wirelessly through the first channel. The medical device system can include a second external device, wherein the second external device can be configured to communicate wirelessly through the second channel. The wireless communications transceiver can be configured to communicate wirelessly to the first external device through the first channel and to communicate wirelessly to the second external device through the second channel.

In a fourteenth aspect, in addition to one or more of the preceding or following aspects, or in the alternative to some aspects, the wireless communications transceiver can be configured to communicate simultaneously through both the first channel and the second channel.

In a fifteenth aspect, in addition to one or more of the preceding or following aspects, or in the alternative to some aspects, the wireless communications transceiver can be configured to troubleshoot the first channel by communicating through the second channel or to troubleshoot the second channel by communicating through the first channel.

In a sixteenth aspect, in addition to one or more of the preceding or following aspects, or in the alternative to some aspects, the DSSS protocol includes encoding communications through the second channel with a spreading sequence such that the communications can only be decoded by devices possessing the spreading sequence.

In a seventeenth aspect, in addition to one or more of the preceding or following aspects, or in the alternative to some aspects, the wireless communications transceiver can be configured to receive commands from the first external device and the second external device, and wherein the control circuit is configured to implement the commands from the second external device but not the first external device.

In an eighteenth aspect, in addition to one or more of the preceding or following aspects, or in the alternative to some aspects, the medical device system includes high-risk data, and wherein the wireless communications transceiver is configured to communicate the high-risk data through only the second channel.

In a nineteenth aspect, a method of wireless communication between an implanted medical device and an external device can be included. The implanted medical device can include a wireless communications transceiver, wherein the wireless communications transceiver can be configured communicate wirelessly through a first channel using a standard BLUETOOTH protocol and communicate wirelessly through a second channel using a DSSS protocol over a BLUETOOTH frequency band. The method can include receiving a first wireless signal from the external device at the wireless communications transceiver. If the first wireless signal is received at the first channel of the wireless communications transceiver, the method can include transmitting a second wireless signal through the first channel to the external device. If the first wireless signal is received at the second channel of the wireless communications transceiver The method can include transmitting a third wireless signal through the second channel to the external device.

In a twentieth aspect, in addition to one or more of the preceding or following aspects, or in the alternative to some aspects, the method can further include transmitting wireless signals simultaneously through both the first channel and the second channel.

In a twenty-first aspect, in addition to one or more of the preceding or following aspects, or in the alternative to some aspects, the method can further include troubleshooting the first channel by communicating through the second channel or troubleshooting the second channel by communicating through the first channel.

In a twenty-second aspect, in addition to one or more of the preceding or following aspects, or in the alternative to some aspects, the method can further include communicating high-risk data through only the second channel.

In a twenty-third aspect, in addition to one or more of the preceding or following aspects, or in the alternative to some aspects, the method can further include communicating through the first channel to one or more patient owned devices and communicating through the second channel to an external medical device.

In a twenty-fourth aspect, in addition to one or more of the preceding or following aspects, or in the alternative to some aspects, the first wireless signal includes a command to be implemented by the implanted medical device, and wherein the implanted medical device can be configured to implement the command if the communications are received over the second channel, and not implement the command if the communications are received over the first channel.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of components of a medical device system in accordance with various embodiments herein.

FIG. 2 is a schematic view of components of a medical device is shown in accordance with various embodiments herein.

FIG. 3 is a schematic view of components of a medical device system in accordance with various embodiments herein.

FIG. 4 is a schematic view of a DSSS transceiver in accordance with various embodiments herein.

FIG. 5 is a schematic view of a signal modulated using a DSSS protocol in accordance with various embodiments herein.

FIG. 6 is a schematic view of a DSSS transceiver in accordance with various embodiments herein.

FIG. 7 is a schematic view of a wireless communications transceiver in accordance with various embodiments herein.

FIG. 8 is a schematic view of a wireless communications transceiver in accordance with various embodiments herein.

FIG. 9 is a schematic view of components of a medical device system in accordance with various embodiments herein.

FIG. 10 is a schematic view of components of a medical device system in accordance with various embodiments herein.

FIG. 11 is a method of wireless communication between an implanted medical device and an external device in accordance with various embodiments herein.

FIG. 12 is a schematic diagram of components of an implantable medical device in accordance with various embodiments herein.

FIG. 13 is a block diagram of components of an implantable medical device in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Wireless communication between an implanted medical device (IMD) implanted in a patient and an external device can be useful to provide monitoring information, provide therapy information, provide programming instructions to the implantable medical device, and for many other purposes. Medical devices may communicate with one or more external devices using standard BLUETOOTH protocols. Utilizing BLUETOOTH wireless technology is advantageous in that most modern consumer devices are BLUETOOTH compatible. However, communications between implantable medical devices and their associated external devices using BLUETOOTH wireless technology standards can pose several challenges including high levels of attenuation, interference, and security risks. However, such challenges can be mitigated and other goals can be achieved by implementing direct sequence spread spectrum technology over a BLUETOOTH frequency band.

Embodiments herein can include medical device systems. For example, an implantable medical device can be included having a biocompatible housing, a control circuit that can be disposed within the biocompatible housing, and a wireless communications transceiver. The wireless communications transceiver can be configured to communicate wirelessly through a first channel using a standard BLUETOOTH protocol and communicate wirelessly through a second channel using a DSSS protocol over a BLUETOOTH frequency band.

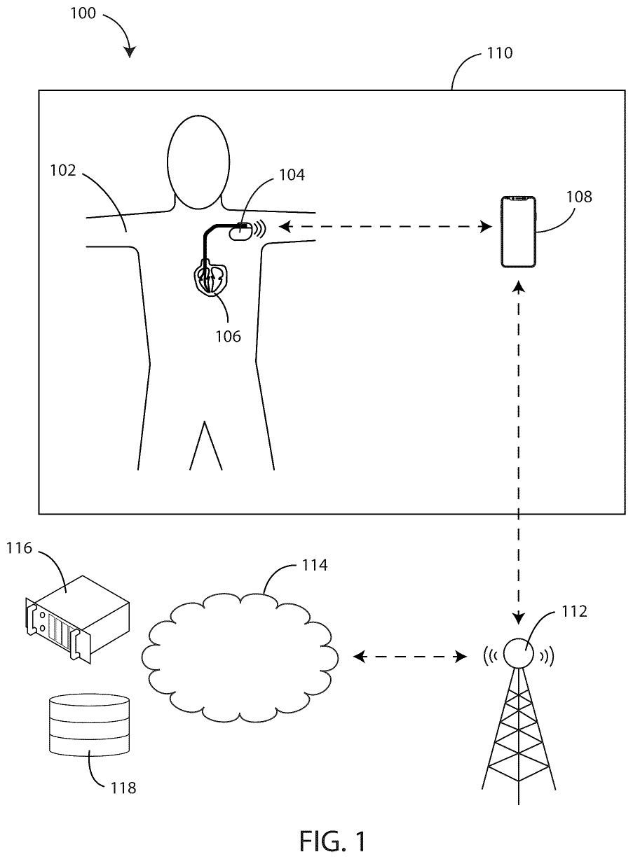

Referring now to FIG. 1, a schematic view of components of a medical device system 100 is shown in accordance with various embodiments herein. The medical device system 100 includes an implantable medical device 104. In various embodiments, the implantable medical device 104 can be any of an implantable monitor, a cardiac rhythm management device, a neuromodulation device, or the like.

The implantable medical device 104 can be implanted within a patient 102 to interface with their heart 106 or another organ. In this example, the medical device system 100 also includes an external device 108. The external device 108 can be, for example, a smart phone, a mobile computing device, a personal patient communicator, or another type of device for facilitating communication of data from the implantable medical device 104 to a data network. In various embodiments, the implantable medical device can include a wireless communications transceiver to enable secure wireless communications between an implantable medical device 104 and one or more external devices 108. The patient 102, the implantable medical device 104, and the external device 108 can all be within the local environment 110. Communications can pass securely from the local environment 110 to a cell tower 112 as facilitated by the external device 108 and then onto the cloud 114 or another data network. In this example, the cloud 114 can include a server 116 (real or virtual) and a database 118 (real or virtual). However, the cloud 114 can also include other computing resources. While not shown in this view, a clinician can access information regarding the patient 102 through the cloud 114 or another data network.

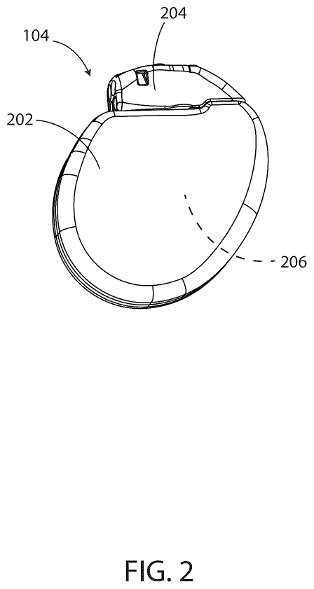

Referring now to FIG. 2, a schematic view of components of a medical device is shown in accordance with various embodiments herein. In various embodiments, the implantable medical device 104 can include a housing 202 and a header structure 204. In some embodiments, housing 202, or one or more portions thereof, can be formed from a biocompatible material including, but not limited to metals, polymers, ceramics, and the like. In some embodiments, one or more segments of the housing 202 can be hermetically sealed. In various embodiments, the implantable medical device can include a wireless communications transceiver 206 to enable secure wireless communications between an implantable medical device 104 and one more external devices 108. Wireless communications transceiver 206 can be disposed within the housing 202 or header structure 204. In some embodiments, the implanted medical device can include a ceramic window, such as a ceramic window on the housing 202 (e.g., defining a portion of the housing) which may otherwise be metal, and wireless communications transceiver 206 can be positioned such that communications can pass through the ceramic window.

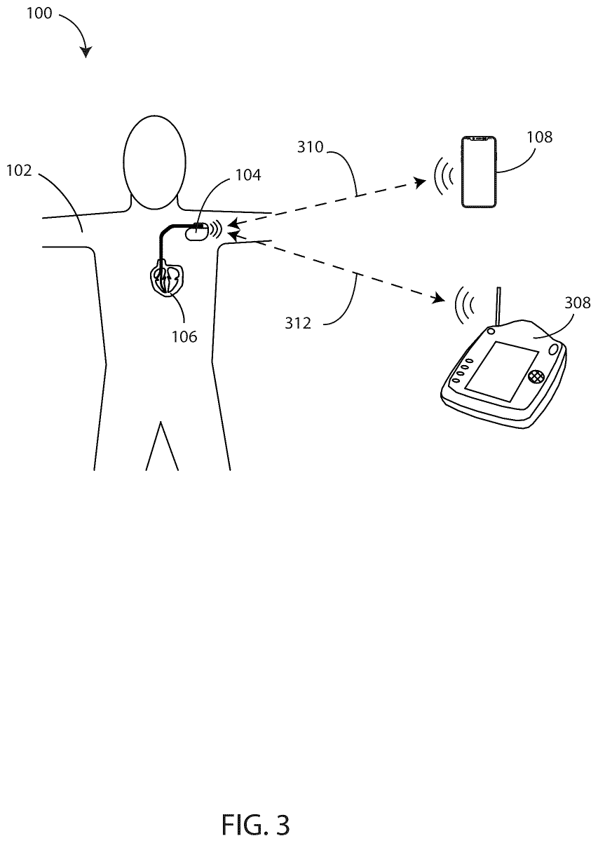

Referring now to FIG. 3, a schematic view of components of a medical device system 100 is shown in accordance with various embodiments herein. The medical device system 100 includes an implantable medical device 104 implanted within a patient 102 to interface with their heart 106 or another organ. In various embodiments, the implantable medical device can include a wireless communications transceiver 206 to enable secure wireless communications between an implantable medical device 104 and one or more external devices. In the example of FIG. 3, the implantable medical device is configured to communicate wirelessly with at least a first external device 108 and a second external device 308. However, the implantable medical device 104 can be configured to communicate with any number of compatible external devices.

In various embodiments, the wireless communications transceiver 206 is configured to communicate using one or more wireless protocols. Wireless frequencies used for ongoing wireless communication can vary, but in some embodiments can include frequencies ranging from 3 kHz to 300 GHz, such as frequency bands including one or more of 900 MHz, 2.4 GHz, 3.6 GHz, 4.9 GHz, 5 GHz, 5.9 GHz, 6 GHz and 60 GHz. In various embodiments, an ISM band can be used at 2.4 to 2.485 GHZ. In some embodiments, the communications can follow any of a secure BLUETOOTH protocol (or variants thereof like BLUETOOTH LOW ENERGY), a secure WIFI protocol, Zigbee wireless communication protocols or another wireless protocol supporting secure communications.

The wireless communications transceiver 206 can communicate over any suitable number of channels, each channel operating with a different wireless communication protocol. In some embodiments, the wireless communications transceiver 206 can communicate over 1, 2, 3, 4, or more channels. In the example of FIG. 3, wireless communications transceiver 206 is configured to communicate over a first channel 310 and a second channel 312. In a particular example, the wireless communications transceiver 206 is configured to communicate wirelessly through the first channel 310 using a standard BLUETOOTH protocol and to communicate wirelessly through a second channel 312 using a DSSS protocol over a BLUETOOTH frequency band.

In some embodiments, the wireless communications transceiver 206 is configured to communicate simultaneously through multiple communications channels (e.g., both the first channel 310 and the second channel 312). In alternative embodiments, the wireless communications transceiver 206 is configured to communicate through only one communication channel at any given time.

In an embodiment, the wireless communications transceiver is configured to communicate wirelessly to the first external device 108 through the first channel 310. The first external device 108 can be any of a cellular phone, a smart phone, a laptop computer, a tablet-style computer, a desktop computer, and a wearable device, an implantable medical device programmer, an external implantable medical device communicator, an external medical device, or the like. The wireless communications transceiver 206 can communicate wirelessly to the first external device 108 through the first channel 310 using a standard BLUETOOTH protocol. Such a protocol is advantageous because most modern consumer devices are configured to communicate using BLUETOOTH. The wireless communications transceiver is configured to communicate wirelessly to any number of external devices through the first channel 310, including both consumer devices and medical device accessories.

In an embodiment, the wireless communications transceiver is configured to communicate wirelessly to the second external device 308 through the second channel 312. The second external device can be any of an implantable medical device programmer, an external implantable medical device communicator, an external medical device, or the like. The wireless communications transceiver 206 can communicate wirelessly to the second external device 308 through the second channel 312 using a DSSS protocol over a BLUETOOTH frequency band. Such a protocol is applicable to devices certified to communicate with the implantable device. Such devices can be manufactured with a transceiver equipped to transmit and receive DSSS modulated signals.

In a particular embodiment, the implantable medical device 104 is a pulse generator and the wireless communications transceiver 206 is configured to communicate through the first channel 310 to one or more patient owned devices and to communicate through the second channel 312 to any of an implantable medical device programmer or an implantable medical device communications device. In an alternative embodiment, the implantable medical device 104 is a pulse generator and the wireless communications transceiver 206 is configured to communicate through the first channel to one or more patient owned devices and to communicate through the second channel to an external medical device.

Implantable medical devices can use any suitable wireless protocol to exchange data with one or more external devices. A wireless protocol is a specification for a set of communication protocols to standardize wireless communication between devices. Examples include Bluetooth, Bluetooth Low Energy, and Zigbee.

Implantable medical devices can also use the classic BLUETOOTH wireless technology standard to exchange data with one or more external devices. BLUETOOTH operates using ultra high-frequency radio waves in the industrial, scientific, and medical radio bands, from 2.402 gigahertz to 2.480 gigahertz, or from 2.400 to 2.4835 gigahertz including guard bands that are 2 megahertz wide at the bottom end and 3.5 megahertz wide at the top end. BLUETOOTH devices transmit data in packets and transmit each packet on one of 79 designated BLUETOOTH channels. Many versions of BLUETOOTH have been developed and will likely continue to be developed, which are compatible with the systems described herein, including BLUETOOTH versions 4.0, 4.1, 4.2, 5.0, 5.1, and 5.2.

Implantable medical devices can use the BLUETOOTH Low Energy (BLE) wireless technology standard to exchange data with one or more external devices. BLE is a wireless communication technology designed for short-range communication between devices with low power consumption requirements. It is an extension of the classic BLUETOOTH technology but optimized for low energy consumption, making it ideal for battery-powered devices (such as implantable medical devices). BLE employs ultra-high frequency (UHF) radio waves in the 2.45-GHz section of the industrial, scientific, and medical (ISM) bands. Specifically, it uses an 80-MHz frequency band from 2.40 GHz to 2.48 GHz.

BLUETOOTH communications pose several challenges for implantable medical devices. BLUETOOTH signals are attenuated more rapidly as they travel through free space and though human tissue compared to lower frequency communications protocols. Moreover, many devices communicate over BLUETOOTH frequencies, so BLUETOOTH communications are prone to interference.

To mitigate the shortcomings of BLUETOOTH communications, the wireless communications transceiver 206 can modulate the data signals using any suitable technique such as direct sequence spread spectrum (DSSS). DSSS is a modulation technique for wireless communication systems. It is a form of spread spectrum modulation, which spreads the signal bandwidth over a larger frequency range than the original data signal. This spreading technique provides benefits such as increased robustness against interference and improved security.

DSSS spreads the signal by multiplying the data signal with a much higher rate pseudorandom sequence known as the spreading code or spreading sequence. The spreading code is a long binary sequence that is designed to have good autocorrelation and cross-correlation properties. This multiplication process expands the bandwidth of the signal. Due to the wide bandwidth and the pseudo-random nature of the spreading code, DSSS signals are more resistant to narrowband interference. Interfering signals appear as noise over the spread spectrum, and the original signal can still be recovered by the receiver using the same spreading code.

Moreover, using DSSS modulation in the BLUETOOTH frequency band will allow both DSSS and BLUETOOTH communications using the same transmitters, antennas, and receivers making it possible for the implantable medical device to communicate with the patient's personal devices (e.g., cell phones, laptops) using BLUETOOTH while achieving superior communication performance with DSSS based compatible programmers and communicators.

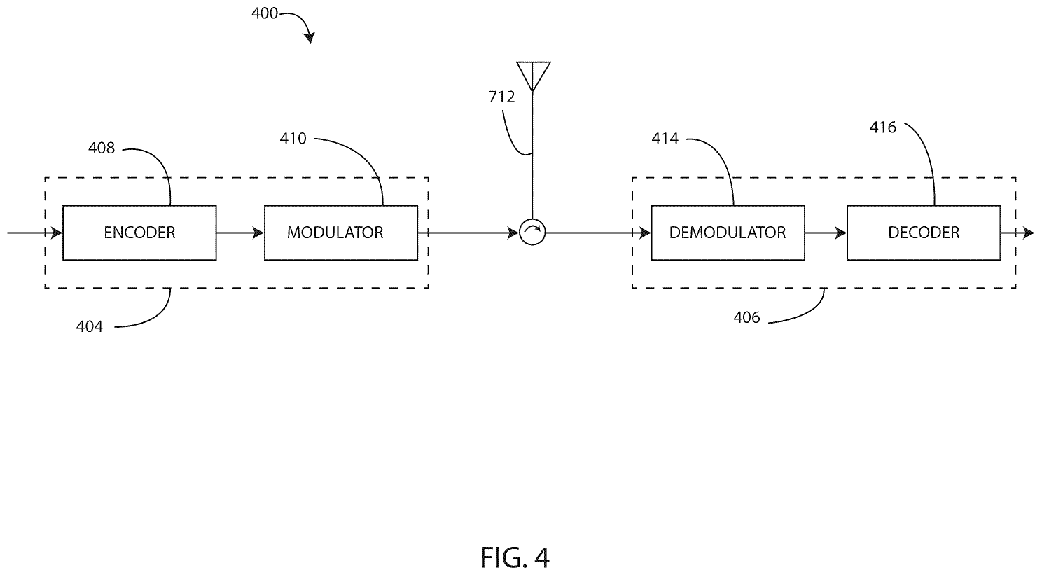

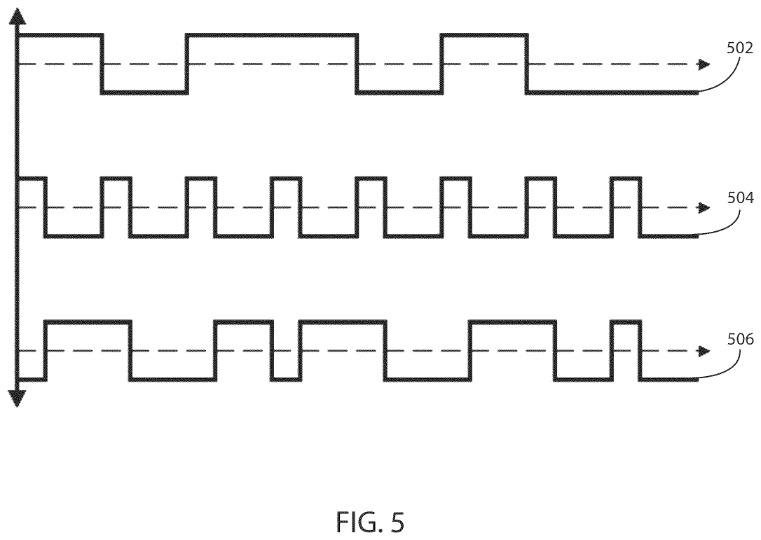

FIGS. 4 and 5 depict an example of a DSSS protocol. FIG. 4 depicts a schematic view of a DSSS transceiver and FIG. 5 depicts a schematic view of a signal modulated using a DSSS protocol in accordance with various embodiments herein. Referring now to FIG. 4, the DSSS system 400 can include a transmitter 404 and a receiver 406. In various embodiments, the transmitter 404 receives input data. Exemplary input data is depicted by input data 502 of FIG. 5. However, the input data can be any suitable type of wireless signal. Upon receiving the input data, transmitter 404 spreads the input data with a spreading signal at the encoder 408. The spreading operation combines the input data bits with a pseudorandom spreading sequence. An example of such a pseudorandom sequence is depicted by pseudorandom sequence 504 of FIG. 5. Because the pseudorandom sequence is designed to resemble white noise, the spectrum of the original signal is spread out. Thus, the spectrum of the spread signal occupies a larger bandwidth.

The transmitter 404 then modulates the encoded signal with a carrier signal at modulator 410. The carrier signal is a high-frequency waveform configured to carry the input from one point to another. The carrier signal acts as a steady reference signal that provides a stable and predictable frequency and amplitude. The carrier signal is typically generated at a much higher frequency than the baseband signal, which represents the actual information being transmitted.

In the example of FIG. 5, after being modulated with pseudorandom sequence 504, the spread signal 506 is spread over a wider range of frequencies than input data 502. The spread signal can then be modulated by the carrier and transmitted through antenna 712.

Antenna 712 can also receive DSSS modulated signals. At the receiver 406, the spread sequence is extracted from the composite signal through demodulation at demodulator 414. The input data is then extracted from the spread signal at decoder 416. The demodulator 414 and decoder 416 reverse the modulation process, separating the carrier from the modulated signal and extracting the encoded data for further processing or playback. This allows for the original input data to be recovered and utilized.

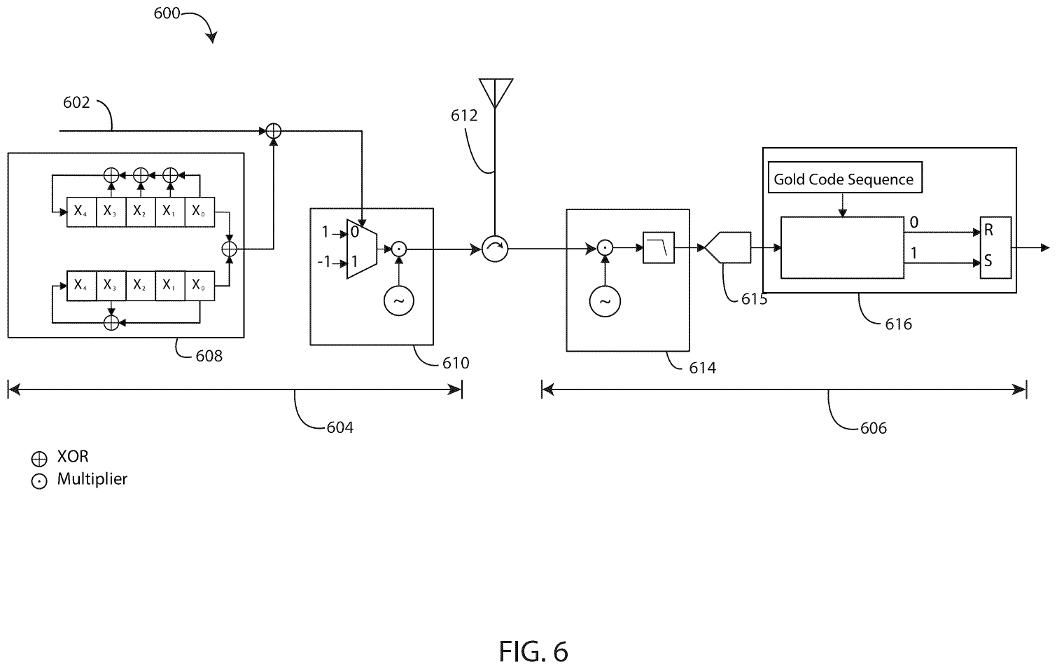

Referring now to FIG. 6, a schematic view of a DSSS transceiver is shown in accordance with various embodiments herein. The DSSS 600 transceiver is configured to communicate wirelessly using a DSSS protocol over a BLUETOOTH frequency band (or may operate over any other suitable wireless frequency band). In various embodiments, the DSSS transceiver includes a transmitter 604 having a DSSS encoder 608 and a data modulator 610, an antenna 612, and a receiver 606 having a base band demodulator 614, an analog to digital converter 615, and a correlation filter 616.

In various embodiments, input data 602 is encoded by a pseudorandom spreading sequence at DSSS encoder 608. In some embodiments, to allow for multiple systems to transmit at the same time over the same spectrum without interference, a “Gold code” pseudorandom spreading sequence can be used. A Gold code sequence is a type of binary sequence used in telecommunication and satellite navigation.

Gold codes are generated by combining two linear feedback shift registers (LFSRs) with different feedback taps and initial states. The two sequences are added modulo-2 (XORed) to generate the final Gold code sequence.

The properties of Gold codes make them useful in wireless communications systems because they exhibit low cross-correlation between different codes, allowing multiple users to share the same bandwidth without significant interference. Additionally, the autocorrelation properties of Gold codes are such that they can be easily synchronized at the receiver, enabling the extraction of the desired user's signal.

In the example of FIG. 6, DSSS encoder 608 contains two linear feedback shift registers to generate sequences that are XORed together to generate a gold code. Each register is initialized with a seed value. Each register can be a 5-cell generator that produces 31-bit sequences. Each bit generated by the Gold code generator is used to modulate a single chip.

After the input data 602 is encoded by a pseudorandom spreading sequence at the DSSS encoder, the data modulator 610 uses the encoded data to select the polarity of the carrier to transmit. The data modulator 610 of FIG. 6 contains a multiplexer that transmits one polarity of the carrier to transmit a 0, and the opposite polarity to transmit a 1.

Modulated signals can be transmitted and received by antenna 612. Signals received at antenna 612 can be demodulated at receiver 606. The carrier signal can be extracted from the received signal at base band demodulator 614. In some embodiments, the received signal is converted to a digital signal at analog to digital converter 615. The received signal is then sent through a correlation filter 616. In various embodiments, the correlation filter possesses the spreading sequence (e.g., the same Gold code) as the DSSS encoder 608. Correlation filter 616 is configured to search for correlation between the received signal and the pseudorandom spreading sequence. In one embodiment, a matching correlation will result in a positive peak for a transmitted one and a negative peak for a transmitted zero (or visa vera). After demodulation, the original signal can be recovered.

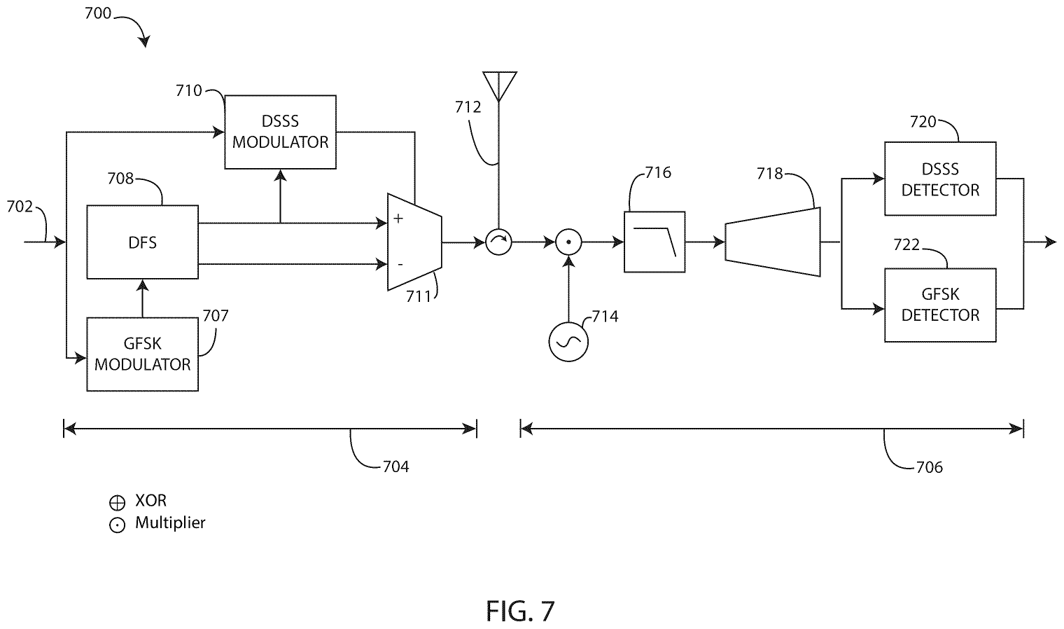

Referring now to FIG. 7, a schematic view of a wireless communications transceiver is shown in accordance with various embodiments herein. In various embodiments, wireless communications transceiver 700 is configured to be disposed in an implantable medical device 104. In various embodiments, the wireless communications transceiver 700 includes a transmitter 704 and a receiver 706 configured to communicate wirelessly through a first channel using a standard BLUETOOTH protocol and communicate wirelessly through a second channel using a DSSS protocol over a BLUETOOTH frequency band.

To transmit a BLUETOOTH signal, the input data 702 is first modulated with a Gaussian frequency-shift modulator (707). Gaussian frequency-shift keying (GFSK) is a type of FSK modulation typically used in BLUETOOTH communications, which uses a Gaussian filter to shape the signal pulses before they are modulated. This reduces the spectral bandwidth and out-of-band spectrum to meet adjacent-channel power rejection requirements. After passing through the Gaussian frequency-shift modulator (707), the signal is then modulated with a carrier signal at Digital frequency synthesizer 708.

To transmit a signal with a DSSS protocol over a BLUETOOTH frequency band, the input data 702 is first modulated with a DSSS modulator 710. DSSS modulator 710 can modulate the input data with any suitable pseudorandom spreading sequence, such as one or more Gold codes as described in the context of FIG. 7. The signal is then modulated with a carrier signal at Digital frequency synthesizer 708.

The BLUETOOTH or DSSS signals can then be passed through phase selector 711 and transmitted via antenna 712. Signals received at antenna 712 can be demodulated at receiver 706. The carrier signal can be extracted from the received signal at base band demodulator 714. In some embodiments, the received signal is then converted to a digital signal at the analog-to-digital converter 718. If the received signal is a standard BLUETOOTH signal (with no DSSS modulation), the signal is demodulated at GFSK detector 722. If the received signal is a DSSS modulated signal, the signal is demodulated at DSSS detector 720. After demodulation, the original signal can be recovered.

As both the standard BLUETOOTH and DSSS communications channels share the same radio frequency (RF) spectrum, the wireless communications transceiver 700 can share many of the same components between the BLUETOOTH and DSSS channels. Starting with a standard BLUETOOTH transceiver circuit, DSSS capability can be added to a transceiver with the addition of a phase selector 711, DSSS modulator 710, and DSSS detector 720. The digital frequency synthesizer 708, RF filters 716 (all not shown in this view), antenna 712, RF amplifiers (not shown), base band demodulator 714, and analog-to-digital converter 718 are shared between both channels.

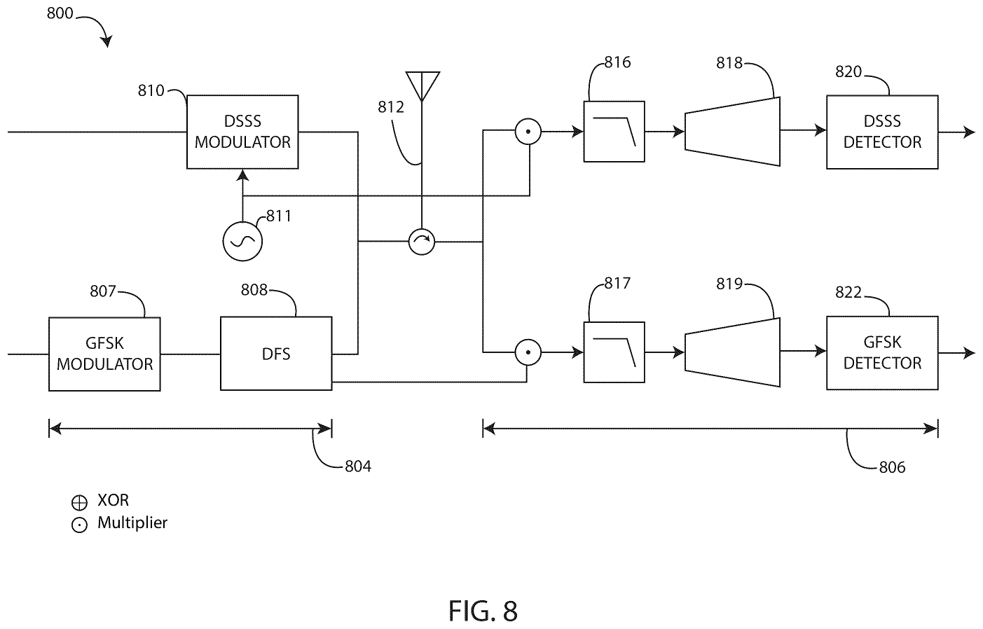

While the wireless communications transceiver 700 of FIG. 7, is configured to communicate using both DSSS and standard BLUETOOTH separately, it is not capable of communicating over both channels simultaneously. Simultaneous usage of both BLUETOOTH and DSSS communication is possible by separating the carrier generators and analog to digital converters as depicted by FIG. 8.

Referring now to FIG. 8, a schematic view of a wireless communications transceiver is shown in accordance with various embodiments herein. In various embodiments, wireless communications transceiver 800 is configured to be disposed in an implantable medical device and is configured to communicate wirelessly through a first channel using a standard BLUETOOTH protocol and to communicate wirelessly through a second channel using a DSSS protocol over a BLUETOOTH frequency band. The wireless communications transceiver 800 operates similarly to wireless communications transceiver 700 but contains additional components to permit simultaneous communications through both standard BLUETOOTH and DSSS channels.

The transmitter is configured to receive input data. To transmit a BLUETOOTH signal, the input data is first modulated with a Gaussian frequency-shift (GFSK) modulator 807. The signal is then modulated with a carrier signal at Digital frequency synthesizer 808. To transmit a DSSS modulated BLUETOOTH signal, the input data is first modulated with a DSSS modulator 810. DFS modulator can modulate the input data with any suitable pseudorandom spreading sequence, such as one or more Gold codes as described in the context of FIG. 6. The signal is then modulated with a carrier signal at DSSS carrier generator 811.

The BLUETOOTH and DSSS signals can then be transmitted (simultaneously or separately) via antenna 812. Signals received at antenna 812 can be demodulated at receiver 806. If the received signal is a BLUETOOTH signal (with no DSSS modulation), the carrier signal can be extracted from the received signal at Digital frequency synthesizer 808 (shared with the transmitter 804 in the example of FIG. 8). In some embodiments, the received signal is then passed though RF filter 817 and converted to a digital signal at analog to digital converter 819. The signal is then demodulated at GFSK detector 822, recovering the original BLUETOOTH modulated data.

If the received signal is modulated with DSSS, the carrier signal can be extracted from the received signal at DSSS carrier generator 811 (shared with the transmitter 804 in the example of FIG. 8). In some embodiments, the received signal is then passed though RF filter 816 and converted to a digital signal at analog to digital converter 818. The signal is demodulated at DSSS detector 820, recovering the original DSSS modulated data.

DSSS modulation offers several advantages. Firstly, DSSS typically expands the signal bandwidth by a factor called the spreading factor. For example, if the spreading factor is 4, the bandwidth of the transmitted signal is four times wider than the original data signal. This expansion allows multiple DSSS signals to coexist without interfering with each other or with narrowband signals.

Moreover, due to the wide bandwidth and the pseudo-random nature of the spreading code, DSSS signals are more resistant to narrowband interference. Interfering signals appear as noise over the spread spectrum, and the original signal can still be recovered by the receiver using the same spreading code.

Additionally, DSSS allows for higher data rates compared to narrowband modulation techniques since the spreading code can be clocked at a much higher rate than the original data signal. The data rate is determined by the spreading factor and the clock rate of the spreading code.

Finally, DSSS offers a certain level of security since the transmitted signal is spread over a wide bandwidth using a specific spreading code. The receiver must know the correct spreading code to successfully demodulate and recover the original data signal. This property makes it difficult for unauthorized receivers to intercept and recover the transmitted information. The security advantages of DSSS will be explained in greater detail herein.

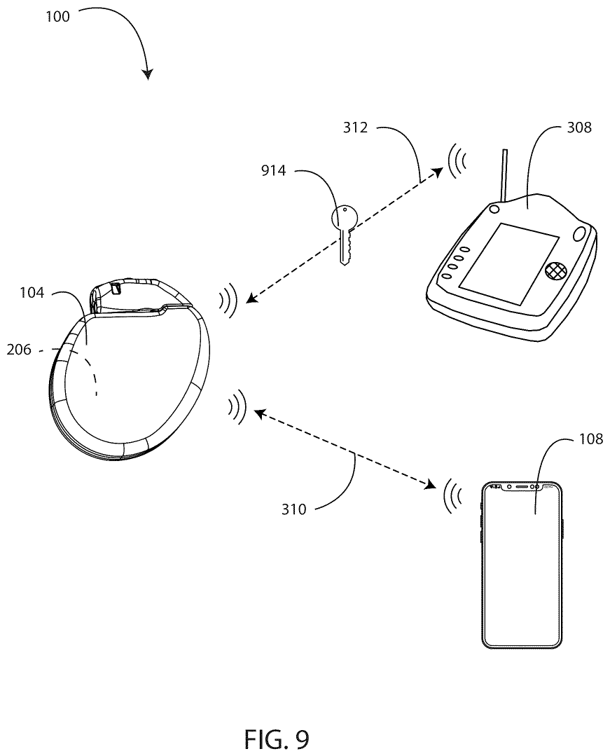

Referring now to FIG. 9, a schematic view of components of a medical device system 100 is shown in accordance with various embodiments herein. The medical device system 100 includes an implantable medical device 104 having a wireless communications transceiver 206 to enable secure wireless communications between an implantable medical device 104 and one or more external devices. In the example of FIG. 9, the implantable medical device is configured to communicate wirelessly with at least a first external device 108 and a second external device 308.

In an embodiment the wireless communications transceiver 206 can communicate wirelessly to the first external device 108 through the first channel 310 using a standard BLUETOOTH protocol and communicate wirelessly to the second external device 308 through the second channel 312 using a DSSS protocol over a BLUETOOTH frequency band.

Encoding signals with a DSSS spreading sequence can be advantageous from a data security perspective. DSSS enhances security since the transmitted signal is spread over a wide bandwidth using a specific spreading code. The receiver must know the correct spreading code to successfully demodulate and recover the original data signal. This property makes it difficult for unauthorized receivers to intercept and understand the transmitted information.

Another added security feature inherent to DSSS is related to establishing the values to use in the spreading sequence (e.g., the Gold code registers such as the ones shown and described in described in the context of FIG. 6). Both the DSSS modulator and demodulator must be programmed with the same values for the signals to be accurately communicated. At the time of manufacture, the implantable medical device and compatible external devices can all be constructed with transceivers having the same Gold code (or other spreading sequence). In an embodiment, a first register of the Gold code generator can be a secret key and a second register of the Gold code generator can be the serial number of the device. Accordingly, the implantable device can receive signals from and identify the compatible external device and vis versa.

In some embodiments, the DSSS protocol includes encoding communications through the second channel with a spreading sequence at the wireless communications transceiver 206 such that the communications can only be decoded by external device(s) possessing the same spreading sequence. The spreading sequence can serve as a security key, such that only communications possessing the security key can perform authorized functions on the implantable medical device 104 (e.g., changing an operating parameter on the implantable medical device or receiving data from the implantable medical device). In the example of FIG. 9, communications through the first channel 310 (without DSSS modulation) are not coded with the security key 914, while communications through the second channel 312 (having DSSS modulation) possess the security key 914.

Such a protocol aids in securing the implantable medical device 104. For example, the wireless communications transceiver is configured to receive the communications from an external device. The communications can include a command (such as a command to change or initiate a device operational state, a command to turn the device off or on, a command to initiate or cease therapy, a command to change or otherwise set an operational or therapy parameter, and the like) to be implemented by the implantable medical device. A control circuit of the implantable medical device can be configured to implement the command if the communications are encoded with the spreading sequence and to ignore or otherwise not implement the command if the communications are not encoded with the spreading sequence. In another example, the control circuit is configured to implement the command if the communications are received over the second channel and to ignore or otherwise not implement the command if the communications are received over the first channel. Such a configuration allows for the implantable medical device to implement commands from trusted certified devices (having the DSSS spreading sequence) while ignoring commands from less reliable external devices.

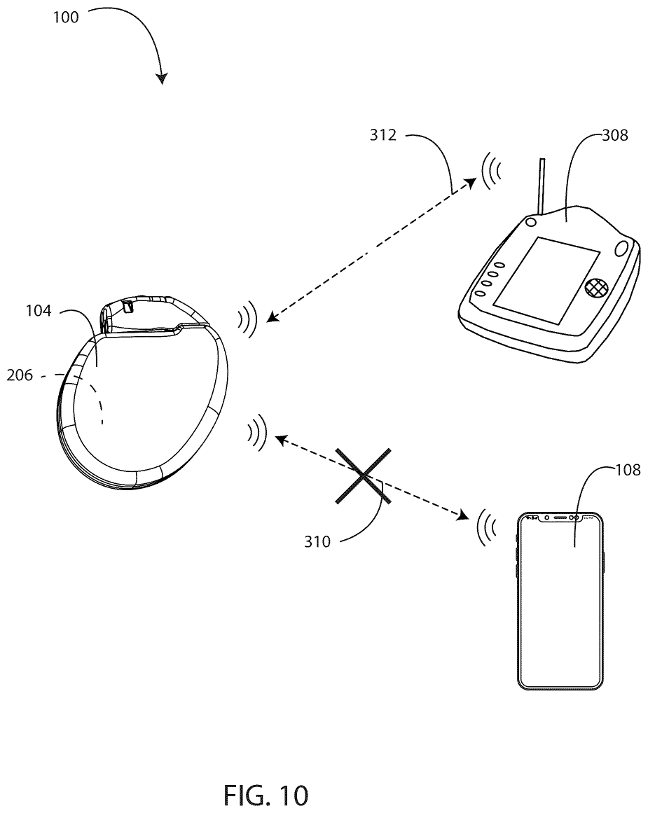

Referring now to FIG. 10, a schematic view of components of a medical device system 100 is shown in accordance with various embodiments herein. The medical device system 100 includes an implantable medical device 104 having a wireless communications transceiver 206 to enable secure wireless communications between an implantable medical device 104 and one or more external devices. In the example of FIG. 10, the implantable medical device is configured to communicate wirelessly with at least a first external device 108 and a second external device 308.

In an embodiment the wireless communications transceiver 206 can communicate wirelessly to the first external device 108 through the first channel 310 using a standard BLUETOOTH protocol and communicate wirelessly to the second external device 308 through the second channel 312 using a DSSS protocol over a BLUETOOTH frequency band.

By utilizing a wireless communications transceiver having two channels, it is possible to only allow certain high-risk communications to occur over DSSS channel and to prevent said high risk communications over the BLUETOOTH channel. Examples of such high-risk communications for an implantable medical device might include commands that change the behavior of the implanted device, turning therapy on or off, changing therapy parameters, or commands that read diagnostic data from the device. In the embodiment of FIG. 10, wireless communications transceiver 206 is configured to communicate high-risk data through the second channel 312, but not the first channel 310.

The multiple channel wireless communications transceiver 206 also has advantages in the context of trouble shooting. Such advantages are especially relevant in wireless communications transceivers that are configured to communicate over both channels simultaneously (such as the wireless communications transceiver 700 of FIG. 7). In various embodiments, the first channel 310 could be used as a diagnostic channel for the second channel 312 (or vis versa). In various embodiments, the wireless communications transceiver is configured to troubleshoot the first channel by communicating through the second channel or to troubleshoot the second channel by communicating through the first channel. For instance, if the wireless communications transceiver is unable to communicate to an external device using DSSS, it can switch to a standard BLUETOOTH protocol, or vis versa. For example, to troubleshoot problems with BLUETOOTH communications, the DSSS channel could provide diagnostic data back to a communicator or programmer. This data might then be used to provide feedback to adjust settings or reset the BLUETOOTH device.

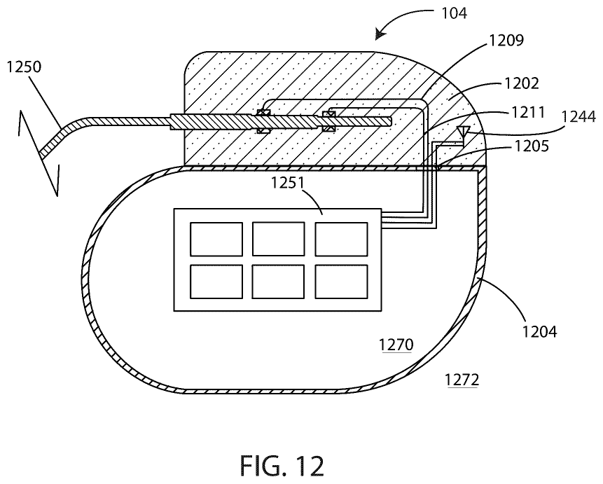

Referring now to FIG. 12, a schematic diagram of components of an implantable medical device is shown in accordance with various embodiments herein. In this example, the implantable medical device 104 includes a header assembly 1202 and a housing 1204. The housing 1204 of the implantable medical device 104 can include various materials such as metals, polymers, ceramics, and the like. In one embodiment, the housing 1204 is formed of titanium. The header assembly 1202 can be coupled to one or more electrical stimulation leads 1250. The header assembly 1202 serves to provide fixation of the proximal end of one or more leads and electrically couples the leads to components within the housing 1204. The header assembly 1202 can be formed of various materials including metals, polymers, ceramics, and the like.

The housing 1204 defines an interior volume 1270 that is hermetically sealed off from the volume 1272 outside of the implantable medical device 104. Various electrical conductors 1209, 1211 can pass from the header assembly 1202 through a feed-through structure 1205, and into the interior volume 1270. As such, the conductors 1209, 1211 can serve to provide electrical communication between the electrical stimulation lead 1250 and circuitry 1251 disposed within the interior volume 1270 of the housing 1204. The circuitry 1251 can include various components such as a microprocessor or control circuit, memory (such as random-access memory (RAM) and/or read only memory (ROM)), a telemetry module (which can include a telemetry antenna), electrical field sensor and stimulation circuitry, a power supply (such as a battery), and a sensor interface channel, amongst others.

The implantable medical device 104 can also include an antenna 1244 that can be disposed within the housing 1204 or, as shown in FIG. 12, within the header assembly 1202. In various embodiments, this antenna can used as part of a first channel to facilitate short range wireless communications, such as can be used herein to pass data which is then used to establish secure ongoing wireless communications through a second channel, such as with the wireless communications transceiver described previously.

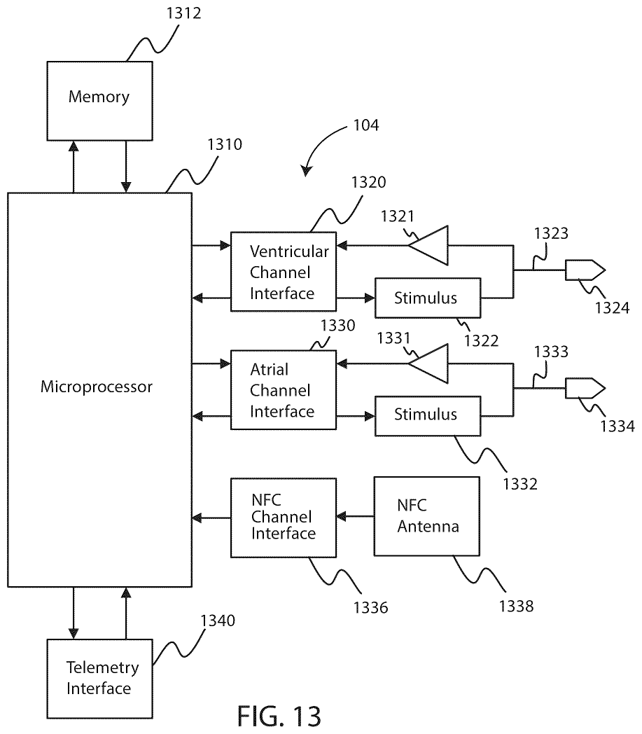

Referring now to FIG. 13, a block diagram of components of an implantable medical device is shown in accordance with various embodiments herein. However, it will be appreciated that some embodiments can include additional elements beyond those shown in FIG. 13. In addition, some embodiments may lack some elements shown in FIG. 13. The implantable medical device of FIG. 13 is a cardiac rhythm management device, but other types of implantable medical devices are also included herein.

The implantable medical device 104 can sense cardiac events through one or more sensing channels and outputs pacing pulses to the heart via one or more pacing channels in accordance with a programmed pacing mode. A microprocessor 1310 can be part of a control circuit and can communicate with a memory 1312 via a bidirectional data bus. The memory 1312 typically comprises read only memory (ROM) or random-access memory (RAM) for program storage and RAM for data storage.

The implantable medical device can include atrial sensing and pacing channels comprising at least a first electrode 1334, lead 1333, sensing amplifier 1331, output circuit 1332, and an atrial channel interface 1330 which can communicate bidirectionally with a port of microprocessor 1310. In this embodiment, the device also has ventricular sensing and pacing channels comprising at least a second electrode 1324, lead 1323, sensing amplifier 1321, output circuit 1322, and ventricular channel interface 1320. For each channel, the same lead and electrode are used for both sensing and pacing. The channel interfaces 1320 and 1330 include analog-to-digital converters for digitizing sensing signal inputs from the sensing amplifiers and registers which can be written to by the control circuitry in order to output pacing pulses, change the pacing pulse amplitude, and adjust the gain and threshold values for the sensing amplifiers.

The implantable medical device can also include a radio frequency antenna 1338 (or another type of wireless communication antenna) and a radio frequency channel interface 1336. A telemetry interface 1340 can be provided for communicating with an external device, such as an external device herein or another type of external device like a programmer. Thus, in this example the radio frequency antenna 1338 (part of a first channel) can be used for providing data used to setup secure communications between the implanted device and the external device which is then conducted over the telemetry interface (part of a second channel).

As described above, the data provided by the wireless communications transceiver can facilitate secure communications between the implantable medical device and the external device(s). The data can be in the form of a password, a token, a key, a digital certificate, or other code or characters. Various shared key authentication techniques can be used including, but not limited to, wired equivalent privacy (WEP), WPA, WPA2, WPA3, or the like. Various pairing and/or handshake protocols can be used including 3-way handshakes, 4-way handshakes, or the like. For example, the protocol can include one or more of a probe request and response, an authentication request and response, and an association request and response. After establishing a secure connection, the ongoing wireless communications can include data that is encrypted by using methods such as temporal key integrity protocol (TKIP), advanced encryption standard (AES), Galois/Counter mode protocol (GCMP), or the like. In some embodiments, the communications can follow a secure BLUETOOTH protocol (or variants thereof like BLUETOOTH LOW ENERGY), a secure WIFI protocol, or another wireless protocol supporting secure communications. Wireless frequencies used for ongoing wireless communication can vary, but in some embodiments can include frequencies ranging from 3 kHz to 300 GHz, such as frequency bands including one or more of 900 MHz, 2.4 GHz, 3.6 GHz, 4.9 GHz, 5 GHz, 5.9 GHz, 6 GHz and 60 GHz. In various embodiments, an ISM band can be used at 2.4 to 2.485 GHZ.

In some embodiments, the security data needed to establish secure communications can also be used for other purposes. For example, in some embodiments, the security data can be used to authorize/enable specific functionality on the part of the external device. In some embodiments, the security data can be used to unlock certain functionality of the external device (for example putting the implanted device into an MRI-safe operating mode). In some embodiments, the security data can be used to cause the implanted medical device to accept a predetermined set of programming commands (such as putting the implanted device into an MRI-safe operating mode) from the external device.

### Methods

Many different methods are contemplated herein, including, but not limited to, methods of making, methods of using, and the like. Aspects of system/device operation described elsewhere herein can be performed as operations of one or more methods in accordance with various embodiments herein.

In various embodiments, operations described herein, and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

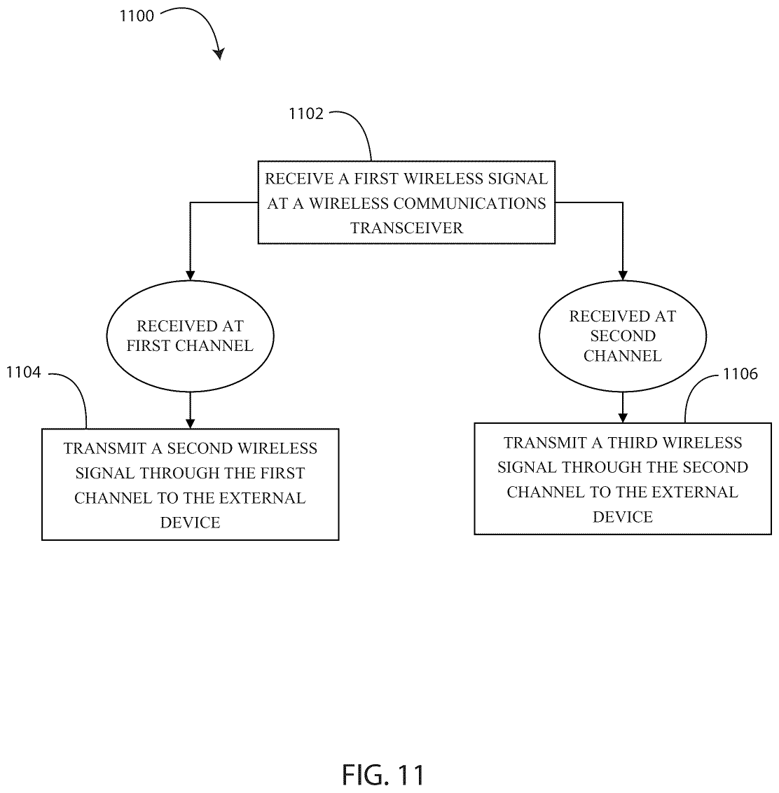

Referring now to FIG. 11, a method of wireless communication between an implanted medical device and an external device is shown in accordance with various embodiments herein. Method 1100 can include step 1102 of receiving a first wireless signal from the external device at the wireless communications transceiver. As described throughout the application the implanted medical device can include a wireless communications transceiver that communicate wirelessly through a first channel using a standard BLUETOOTH protocol (non-DSSS) and communicate wirelessly through a second channel using a DSSS protocol over a BLUETOOTH frequency band.

If the first wireless signal is received at the first channel of the wireless communications transceiver, the method 1100 can include the step 1104 of transmitting a second wireless signal through the first channel to the external device. External devices that communicate to the wireless transceiver through the first channel can include a smart phone, a laptop computer, a tablet-style computer, a desktop computer, and a wearable device, or the like.

If the first wireless signal is received at the second channel of the wireless communications transceiver, the method 1100 can include the step 1106 of transmitting a third wireless signal through the second channel to the external device. External devices that communicate to the wireless transceiver through the second channel can include an implantable medical device programmer, an external implantable medical device communicator, and an external medical device, or any device able to decode the DSSS protocol.

In an embodiment, the method can further include transmitting wireless signals simultaneously through both the first channel and the second channel.

In an embodiment, the method can further include troubleshooting the first channel by communicating through the second channel or troubleshooting the second channel by communicating through the first channel.

In an embodiment, the method can further include communicating high-risk data through only the second channel.

In an embodiment, the method can further include communicating through the first channel to one or more patient owned devices and communicating through the second channel to an external medical device.

In an embodiment of the method, the first wireless signal comprises a command to be implemented by the implanted medical device, and wherein the implanted medical device is configured to: implement the command if the communications are received over the second channel, and not implement the command if the communications are received over the first channel.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An implantable medical device comprising:
a biocompatible housing;
a control circuit, wherein the control circuit is disposed within the biocompatible housing; and
a wireless communications transceiver;
wherein the wireless communications transceiver is configured to
communicate wirelessly through a first channel using a standard BLUETOOTH protocol; and
communicate wirelessly through a second channel using a direct sequence spread spectrum (DSSS) protocol over a BLUETOOTH frequency band.

2. The implantable medical device of claim 1, wherein the wireless communications transceiver is configured to communicate simultaneously through both the first channel and the second channel.

3. The implantable medical device of claim 1, wherein the wireless communications transceiver is configured to communicate wirelessly to a first external device through the first channel and to communicate wirelessly to a second external device through the second channel.

4. The implantable medical device of claim 3, wherein the first external device is at least one of a cellular phone, a smart phone, a laptop computer, a tablet-style computer, a desktop computer, and a wearable device.

5. The implantable medical device of claim 3, wherein the second external device is at least one of an implantable medical device programmer, an external implantable medical device communicator, and an external medical device.

6. The implantable medical device of claim 1, wherein the wireless communications transceiver is configured to troubleshoot the first channel by communicating through the second channel or to troubleshoot the second channel by communicating through the first channel.

7. The implantable medical device of claim 1, wherein the DSSS protocol includes encoding communications through the second channel with a spreading sequence such that the communications can only be decoded by devices possessing the spreading sequence.

8. The implantable medical device of claim 7, wherein the wireless communications transceiver is configured to receive the communications from an external device, wherein the communications comprise a command to be implemented by the implantable medical device, and wherein the control circuit is configured to implement the command if the communications are encoded with the spreading sequence, and not implement the command if the communications are not encoded with the spreading sequence.

9. The implantable medical device of claim 1, wherein the wireless communications transceiver is configured to receive the communications from an external device, wherein the communications comprise a command to be implemented by the implantable medical device, and wherein the control circuit is configured to implement the command if the communications are received over the second channel, and not implement the command if the communications are received over the first channel.

10. The implantable medical device of claim 1, wherein the implantable medical device comprises high-risk data, and wherein the wireless communications transceiver is configured to communicate the high-risk data through only the second channel.

11. The implantable medical device of claim 1, wherein the implantable medical device is a pulse generator and the wireless communications transceiver is configured to communicate through the first channel to one or more patient owned devices and to communicate through the second channel to any of an implantable medical device programmer or an implantable medical device communications device.

12. The implantable medical device of claim 1, wherein the implantable medical device is a pulse generator and the wireless communications transceiver is configured to communicate through the first channel to one or more patient owned devices and to communicate through the second channel to an external medical device.

13. A medical device system comprising:
an implantable medical device, the implantable medical device comprising
a control circuit; and
a wireless communications transceiver;
wherein the wireless communications transceiver is configured to
communicate wirelessly through a first channel using a standard BLUETOOTH protocol; and
communicate wirelessly through a second channel using a DSSS protocol over a BLUETOOTH frequency band;
a first external device, wherein the first external device is configured to communicate wirelessly through the first channel;
a second external device, wherein the second external device is configured to communicate wirelessly through the second channel; and
wherein the wireless communications transceiver is configured to communicate wirelessly to the first external device through the first channel and to communicate wirelessly to the second external device through the second channel.

14. The medical device system of claim 13, wherein the wireless communications transceiver is configured to communicate simultaneously through both the first channel and the second channel.

15. The medical device system of claim 13, wherein the wireless communications transceiver is configured to troubleshoot the first channel by communicating through the second channel or to troubleshoot the second channel by communicating through the first channel.

16. The medical device system of claim 13, wherein the DSSS protocol includes encoding communications through the second channel with a spreading sequence such that the communications can only be decoded by devices possessing the spreading sequence.

17. The medical device system of claim 13, wherein the wireless communications transceiver is configured to receive commands from the first external device and the second external device, and wherein the control circuit is configured to implement the commands from the second external device but not the first external device.

18. The medical device system of claim 13, wherein the medical device system comprises high-risk data, and wherein the wireless communications transceiver is configured to communicate the high-risk data through only the second channel.

19. A method of wireless communication between an implanted medical device and an external device comprising:
the implanted medical device comprising a wireless communications transceiver;
wherein the wireless communications transceiver is configured communicate wirelessly through a first channel using a standard BLUETOOTH protocol and communicate wirelessly through a second channel using a DSSS protocol over a BLUETOOTH frequency band;
receiving a first wireless signal from the external device at the wireless communications transceiver;
if the first wireless signal is received at the first channel of the wireless communications transceiver: transmitting a second wireless signal through the first channel to the external device; and
if the first wireless signal is received at the second channel of the wireless communications transceiver: transmitting a third wireless signal through the second channel to the external device.

20. The method of claim 19, wherein the first wireless signal comprises a command to be implemented by the implanted medical device, and wherein the implanted medical device is configured to: implement the command if the communications are received over the second channel, and not implement the command if the communications are received over the first channel.
